(19) United States
(12) Patent Application Publication
(10) Pub. No.: US 2021/0260275 A1
(43) Pub. Date: Aug. 26, 2021

(54) MULTI-USE DRUG DELIVERY DEVICE FOR DRUGS WITH INSUFFICINET LEVEL OF PRESERVATIVES

(71) Applicant: Novo Nordisk A/S, Bagsvaerd (DK)

(72) Inventors: Henrik Bengtsson, Taastrup (DK); Vera Pinto Glenting, Copenhagen (DK); Joern Drustrup, Farum (DK)

(21) Appl. No.: 17/251,255

(22) PCT Filed: Jun. 3, 2019
(86) PCT No.: PCT/EP2019/064264

§ 371 Date: Dec. 11, 2020

(30) Foreign Application Priority Data
Jun. 14, 2018 (EP) 18177755.8

(51) Int. Cl. A61M 5/00; A61M 5/28; A61M 5/315; A61M 5/32

(52) U.S. Cl. CPC A61M 5/001; A61M 5/286; A61M 2005/3128; A61M 5/3202; A61M 5/3155

(57) ABSTRACT

A multi-use injection device (100) for multiple subcutaneous injections, wherein the injection device comprises a device main portion (110) and a multi-use needle unit (150) adapted to prevent unintended introduction of living microorganisms into a reservoir (114) of the device main portion during use of the injection device, and thereby promote bacteriostasis of a multiple-use drug preparation (115) during a use period with multiple injections. The drug delivery device (100) is adapted to enable multiple injections, without accidentally introducing living microbial contaminations into the reservoir during use, by adapting the multi-use needle to: (i) sterilize contaminations introduced into the distal needle after each exposure of a distal injection needle (156), (ii) restrict diffusion and flow from the distal needle (156) to the reservoir (114) with a valve (170), (iii), and by allowing the needle unit to remain mounted in the in-use configuration during the use period with multiple injections.

### TECHNICAL FIELD

The present invention relates to a multi-use injection device for extended use, wherein the multi-use injection device comprises a device main portion and a multi-use needle, wherein the device main portion comprises a drug reservoir, wherein the reservoir comprises multiple doses of a liquid drug formulation, and wherein the drug formulation allows microbial growth upon introduction of microorganisms into the reservoir during extended use, i.e., the drug formulation comprise less preservatives and the concentration is insufficient to inhibit microbial growth upon unintended introduction of microbial contamination. The invention further relates to a method of using the multi-use injection device for multiple injections during a use period. The invention further relates to a method of using the multi-use injection device for multiple exposures during a use period. The invention further relates to a method of using the multi-use injection device for multiple injections during a use period, wherein the multi-use needle is permanently engaged with the device main portion during the use period.

### BACKGROUND OF THE INVENTION

In the disclosure of the present invention reference is mostly made to the treatment of diabetes by delivery of liquid insulin formulation, however, this is only an exemplary use of the present invention.

Drug delivery devices in the form of drug injection devices have greatly improved the lives of patients who must self-administer liquid drugs and biological agents. Drug injection devices may take many forms, including simple disposable devices that are little more than an ampoule with an injection means or they may be highly sophisticated electronically controlled instruments with numerous functions. Some devices are intended for single-use and may come with an integrated needle, e.g. comprising a so-called pre-filled syringe. However, in case the drug delivery device is intended to be used for multiple injections, it will typically be designed for use with a replaceable needle or cannula unit which ideally is to be replaced for each injection of a dose of drug. Regardless of their form, they have proven to be great aids in assisting patients to self-administer injectable drugs and biological agents. They also greatly assist care givers in administering injectable medicines to those incapable of performing self-injections.

In particular pen-style injection devices have proven to provide an accurate, convenient, and often discrete, way to administer drugs and biological agents, such as insulin. While pen-style injection devices are typically cylindrically shaped with a mounted needle protruding from the most distal portion of one end of the device, some devices have other shapes with the needle no longer protruding from the most distal part of an end of the device, e.g. Innovo® and InnoLet® from Novo Nordisk A/S, Bagsvrd, Denmark.

Typically, injection devices use a pre-filled cartridge containing the liquid medication of interest, e.g. 1.5 or 3.0 ml of insulin or growth hormone formulation. The cartridge is typically in the form of a generally cylindrical transparent glass cylinder having a distal bottle neck portion with a distal opening closed by a needle pierceable septum and an opposed proximal opening in which an elastomeric piston is received, the piston being arranged to be moved by the dosing mechanism of the injection device. The injection devices generally are of two types: “Durable” devices and “disposable” devices. A durable device is designed to allow a user to replace one cartridge with another cartridge, typically a new cartridge in place of an empty cartridge. In contrast, a disposable device is provided with an integrated cartridge which cannot be replaced by the user; when the cartridge is empty the entire device is discarded.

As described above, a drug delivery device intended to be used for multiple injections is typically designed to be used in combination with a replaceable needle unit comprising a proximal needle portion adapted to be inserted into the drug-filled cartridge through a needle-penetrable septum seal and a distal needle portion adapted to be introduced subcutaneously, this allowing a given dose amount of liquid drug formulation to be injected subcutaneously through the hollow needle. Since the proximal needle portion penetrates the seal of the cannula and provides a flow path from the inside of the cannula to the outside, a risk of contamination of the cartridge contents is introduced.

The risk of contamination is primarily related to removal of the needle unit or the infusion set after use. As long as the cannula is penetrating the cartridge seal, it provides access from surroundings to the drug formulation and should thus be removed immediately after injection. However, after injection but prior to removal of the needle, the small volume of drug formulation inside the needle itself may be contaminated either from body fluids or from bacteria in the surroundings when the cannula is extracted from the skin of the subject. When the cannula is removed from the cartridge, some of the remaining fluid in the cannula may be sucked into the cartridge, thereby contaminating the drug formulation in the cartridge.

Therefore, drug formulations for use in multi dose injection devices must contain a sufficient level of preservatives to insure biostatic conditions during the expected in-use time of the cartridge to counter such contamination, i.e., to ensure conditions wherein growth of microorganisms are inhibited. This requirement is included in chapters on injectable drug formulations in current versions of international pharmacopeia.

The different national and international pharmacopoeias are issued by officially recognized authorities and provide common quality standards throughout the pharmaceutical industry. The standards for product quality tests of parenteral drug products, which include injections, is a part of the pharmacopoeia and some of the requirements are described in the following. Parenteral drug products are injected through the skin or other external boundary tissue, to allow the direct administration of the active drug substance(s) into blood vessels, organs, tissues, or lesions. Injections may exist as either immediate- or extended-release dosage forms. Routes of administration for parenteral drug products include intravenous, intraventricular, intra-arterial, intra-articular, intramuscular, intrathecal, intracisternal, intraocular and subcutaneous. Parenteral dosage forms include solutions, suspensions, emulsions, sterile powders for solutions and suspensions (including liposomes), and products that consist of both a drug and a device such as drug-eluting stents.

A regulatory requirement to drug delivery devices is that the packaging system should not interact physically or chemically with the preparation to alter its strength, quality, or purity beyond the official or established requirements. The packaging system should be closed or sealed in such a manner as to prevent contamination or loss of contents. Validation of container integrity must demonstrate no penetration of microbial contamination or gain or loss of any chemical or physical parameter deemed necessary to protect the product.

The above mentioned drug delivery devices are more than just a packaging system, as they have additional functions to ease administration. Such drug delivery devices may be referred to as dual function container-closure systems.

Closures for multiple-dose containers permit the withdrawal of the contents without removal or destruction of the closure. The closure permits penetration by a needle and, upon withdrawal of the needle, closes at once, protecting the container against contamination. Validation of the multiple-dose container integrity must include verification that such a package prevents microbial contamination or loss of product contents under anticipated conditions of multiple entry and use.

For example, for testing prefilled syringes without attached sterile needles, the test includes expelling and transferring the content to a culture medium. At intervals during the incubation period and at its conclusion, examine the media for macroscopic evidence of microbial growth. If no evidence of microbial growth is found, the product to be examined complies with the test for sterility

In multiple-dose containers the liquid drug is preserved with preservatives in order to prevent microbial growth during the extended use, i.e., small doses over an extended in-use time as in continuous delivery or larger doses over an extended in-use time. The use of preservatives may in some cases reduce the efficacy of the drug and in some cases be incompatible with the drug, which means that such type of drug formulations cannot be used with a multi-dose injection device. For example, the necessary preservatives would destroy the drug substance in the cartridge by precipitating the drug substance or chemically react with it.

WO 2015/177082 discloses a medical cartridge for multiple doses of a medical drug, which allows the waste of medical drug to be minimised, without requiring the use of preservatives in the medical drug. The medical cartridge is provided with a one way valve, arranged in an interior part of the medical cartridge at a position near an outlet end. The one way valve is arranged to allow a fluid flow from the interior of the medical cartridge towards the outlet end, and to prevent a fluid flow from the outlet end towards the interior of the medical cartridge. An injection needle can be mounted via a needle adapter at the outlet end of the cartridge, and extends through a septum, at the outlet end of the cartridge. It is an advantage that the one way valve is arranged in an interior part of the cartridge, because thereby the one way valve can be designed in a manner which reduces a dead volume inside the cartridge. By arranging the one way valve in the interior part of the cartridge, no additional or exterior interface between the outlet end of the cartridge and the one way valve is required, and thereby the risk of leaks at such an interface is eliminated, or at least considerably reduced. The description of a different embodiment indicates that the one way valve may replace a passive septum of the medical cartridge. According to such an embodiment, the one way valve is arranged inside the cartridge, immediately adjacent to the outlet end, and in immediate contact with an injection needle connected to the outlet end of the cartridge. This design may even further reduce the dead volume inside the cartridge, thereby even further reducing the waste of medical drug.

For such systems it is important that there is no introduction of microorganisms during use, which includes the handling steps in connecting and removing a needle unit from the drug delivery device multiple times during the extended use period. Therefore improved injection devices and methods for multiple injections for drugs with insufficient levels of preservatives are needed. Therefore, one of the objectives of the present application is to provide devices and methods adapted to prevent unintended introduction of living micro-organisms into a drug reservoir of the device during use of the injection device. Another object is to provide devices and methods suitable for use with conventional injection devices comprising septum sealed reservoirs.

### DISCLOSURE OF THE INVENTION

In the disclosure of the present invention, embodiments and aspects will be described which will address one or more of the above objects or which will address objects apparent from the below disclosure as well as from the description of exemplary embodiments.

Thus, in a general aspect of the invention is provided a multi-use injection device for multiple subcutaneous injections, wherein the injection device comprises a device main portion and a multi-use needle unit adapted to prevent unintended introduction of living microorganisms into a reservoir of the device main portion during use of the injection device, and thereby promote bacteriostasis of a multiple-use drug preparation during a use period with multiple injections:
the device main portion comprises:
a cartridge comprising the reservoir with the multiple-use drug preparation for subcutaneous injection, wherein the multiple-use drug preparation comprises a mixture of substances that do not prevent growth of microorganisms if accidentally introduced into the reservoir with contaminations during use of the drug delivery device, wherein the cartridge further comprises a rigid cylindrical body with a distal end closed and sealed by a needle-piercable membrane, and a piston slidably inserted in the cylindrical body,
a needle mount enabling the multi-use needle unit to be mounted and establish fluid communication with the reservoir by piercing the needle piercable membrane,
dose setting member adapted to set a variable dose to be expelled after each injection or a fixed dose mechanism to set a fixed dose to be expelled after each injection, an expelling mechanism comprising a piston drive member adapted to drive the piston and thereby pressurize the reservoir and expel the set dose through a mounted multi-use needle unit;
wherein the multi-use needle unit comprises:
a connecting member for mounting the multi-use needle unit on the needle mount,
a proximal hollow needle adapted to penetrate the needle-piercable membrane, wherein the proximal needle is adapted to establish fluid communication with the reservoir, when the needle unit is mounted on the needle mount,
a distal hollow expelling needle with a sharp distal end, wherein the distal needle is in fluid communication with the proximal needle and adapted to be inserted into the subcutaneous tissue of a subject and to provide an outlet for expelling the drug from the reservoir,
a movable shield distally carrying a cleaning member, wherein the shield is adapted to be slidably operated between a first and a second axial position relative to the distal end of the distal needle, wherein (i) the first axial position comprises the shield extending distally to the distal end of the distal needle, whereby the shield covers the distal needle and the distal end is arranged in the cleaning member, wherein (ii) the second axial position comprises the distal end extending distally to the shield, wherein the distal end is exposed and ready for injection into the subcutaneous layer of a subject,
   wherein the cleaning member is adapted to preserve the distal needle in a sterile condition,
   wherein the shield is arranged to automatically return to its first position following exposure of the distal needle, wherein the cleaning member is adapted to sterilize any introduced contaminations during exposure of the distal needle,
a pressure-controlled valve, wherein the valve is adapted to:
   allow a drug flow from the reservoir to the distal needle, in response to pressurization of the reservoir and the establishment of a pressure drop across the valve in a direction from the reservoir towards the distal needle, wherein the pressure drop exceeds a first threshold,
   restrict a drug flow from the distal needle to the reservoir, in response to the establishment of a pressure drop across the valve in the direction from the distal needle towards the reservoir, wherein the pressure drop exceeds a second threshold,
   restrict diffusion from the distal needle to the reservoir, when the pressure drop across the valve is below the second threshold,
wherein the drug delivery device comprises:
   an initial assembled configuration prior to a first exposure of the distal needle, wherein the needle unit is mounted on the needle mount of the device main portion with the proximal needle extending into the reservoir,
   an in-use configuration, wherein the needle unit is mounted on the needle mount of the device main portion with the proximal needle extending into the reservoir, wherein the proximal needle and the distal needle are filled with drug from the reservoir, and wherein drug delivery device is adapted to establish a vacuum in the reservoir and a pressure drop across the valve in the direction from the distal needle towards the reservoir, in response to demounting the needle unit and thereby withdrawing the primed proximal needle from the reservoir,
wherein the drug delivery device is adapted to enable multiple injections, without accidentally introducing living microbial contaminations into the reservoir during use, by adapting the multi-use needle to: (i) sterilize contaminations introduced into the distal needle after each exposure, (ii) restrict diffusion and flow from the distal needle to the reservoir, (iii), and by allowing the needle unit to remain mounted in the in-use configuration during the use period with multiple injections, without risking contamination of the multiple-use drug preparation (115) comprising the mixture of substances that do not prevent growth of microorganisms if the contaminations are accidentally introduced into the reservoir during use of the drug delivery device.

Hereby is provided an injection device with a multi-use needle unit, wherein the needle unit is adapted for multi-use purposes, and thereby takes away the requirement of changing needle. The pressure controlled valve and the cleaning member reduces the effect, if a vacuum accidentally should be established.

In a further aspect, the needle-unit and the device main portion are adapted to irremovably engage, when the needle unit is mounted on the needle mount.

In a further aspect, the cleaning member comprises a cleaning agent adapted to react with microbial contaminations introduced with the distal needle.

In a further aspect, the cleaning member is a solid.

In a further aspect, the cleaning member comprises a cleaning chamber with a liquid cleaning agent.

In a further aspect, the cleaning member comprises m-cresol or phenol.

In a further aspect, the injection device further comprises a cap, wherein the device main portion further comprises a cap mount to receive the cap in a mounted state, wherein the cap is adapted to enclose the needle mount in the mounted state.

In a further aspect, the cap is further adapted to receive and enclose a multi-use needle unit mounted on the needle mount.

In a further aspect, the needle unit further comprises a spring arranged and adapted to automatically return the shield to its first axial position, in response to releasing a force urging the shield towards the second axial position.

In a further aspect, the injection device further comprises a disassembled configuration, wherein the device main portion and the needle unit are separated.

In a further aspect is provided a method of using the multi-use injection device according to any of the previous claims, wherein the injection device comprises the device main portion and the multi-use needle unit adapted to prevent unintended introduction of living microorganisms into a reservoir of the device main portion during use of the injection device, and thereby promote bacteriostasis of a multiple-use drug preparation during a use period with multiple injections, wherein the method comprises:
sterilizing microbial contaminations introduced into the distal needle after each exposure,
restricting diffusion and flow from the distal needle to the reservoir, and
performing multiple injections, wherein the needle unit remains mounted in the in-use configuration during the use period with multiple injections.

In a further aspect, the needle-unit and the device main portion are adapted to irremovably engage, when the needle unit is mounted on the needle mount, wherein the method further comprises mounting the needle unit on the needle mount and thereby permanently engaging the needle unit to the device main portion.

Alternatively or in a further aspect is provided a method using the multi-use injection device according to the above described aspect, wherein the needle-unit and the device main portion are adapted to irremovably engage, when the needle unit is mounted on the needle mount, wherein the method comprises mounting the needle unit on the needle mount and thereby permanently engaging the needle unit to the device main portion.

Alternatively or in another aspect is provided a method of using the multi-use injection device according to any of the above described aspects, wherein the injection device comprises the device main portion and the multi-use needle unit is adapted to prevent unintended introduction of living microorganisms into a reservoir of the device main portion during use of the injection device, and thereby promote bacteriostasis of a multiple-use drug preparation during a use period with multiple injections, wherein the method comprises:
providing the injection device in the initial assembled configuration, wherein the needle unit is mounted on the needle mount of the device main portion with the proximal needle extending into the reservoir,
moving the shield and the cleaning member from the first axial position to the second axial position, whereby the distal end extends distally to the shield, wherein the distal end is exposed to contaminations in the surroundings and is ready for the initial priming and expelling an amount of drug,
pressurizing the reservoir and thereby expelling an initial amount of drug, whereby the proximal needle and the distal needle are filled with drug, whereby the injection device is in the in-use configuration,
moving the shield and the cleaning member from the second axial position to the first axial position, and thereby positioning the distal end of the distal tip in the cleaning member, whereby the cleaning member sterilizes any introduced contamination,

for handling the injection device to provide further expulsions during use, the method further comprises:
a) moving the shield and the cleaning member from the first axial position to the second axial position, whereby the distal end extends distally to the shield, wherein the distal end is exposed to contaminations in the surroundings and is ready for expelling an amount of drug,
b) pressurizing the reservoir and thereby expelling an amount of drug, whereby the proximal and the distal needle are filled with drug, and whereby the expelling releases the pressure in the reservoir until the pressure reaches the first threshold, wherein the pressure controlled valve closes and restricts diffusion and flow from the distal needle to the reservoir,
c) moving the shield and the cleaning member from the second axial position to the first axial position, and thereby positioning the distal end of the distal needle in the cleaning member, whereby the cleaning member sterilizes any introduced contamination,
whereby the method provides multiple injections, without accidentally introducing living microbial contamination into the reservoir during use, by (i) sterilizing microbial contaminations introduced into the distal needle after each exposure, (ii) restricting diffusion and flow from the distal needle to the reservoir, (iii), and performing multiple injections, wherein the needle unit remains mounted in the in-use configuration during the use period with multiple injections.

### BRIEF DESCRIPTION OF THE DRAWINGS

In the following embodiments of the invention will be described with reference to the drawings:

FIGS. 1A, 1B show an example of a drug delivery device in the form of an injection device. The injection device is shown with and without a protecting cap.

FIG. 1C shows a pressurizable drug reservoir in the form of a cartridge, which can be inserted into the drug delivery device shown in FIGS. 1A and 1B

FIG. 2 show an injection device with a multi-use needle according to the present disclosure.

FIGS. 3A-3C show details of the working principle of an embodiment of the pressure controlled valve in the multi-use needle illustrated in FIG. 2.

FIGS. 4A-4E show a sequence of device manipulations during an injection with the injection device illustrated in FIG. 2, and thereby collectively illustrate the working principle of expelling or injecting a drug.

In the figures like structures are mainly identified by like reference numerals.

### DESCRIPTION OF EXEMPLARY EMBODIMENTS

When in the following terms such as “upper” and “lower”, “right” and “left”, “horizontal” and “vertical” or similar relative expressions are used, these only refer to the appended figures and not necessarily to an actual situation of use. The shown figures are schematic representations for which reason the configuration of the different structures as well as their relative dimensions are intended to serve illustrative purposes only. When the term member is used for a given component it can be used to define a unitary component or a portion of a component, having a one or more functions.

Before turning to embodiments of the present invention per se, an example of an automatic drug delivery device in the form of an automatic injection device for multiple injections will be described. The described drug delivery devices provide the basis for the exemplary embodiments of the present invention.

The automatic drug delivery device is a prior art resettable dial-up/dial down automatic drug delivery device will be described. The pen device 1 comprises a cap part 42 and a device main portion 10 having a proximal body or drive assembly portion with a housing 2 in which a drug expelling mechanism is arranged or integrated, and a distal cartridge holder portion 46 in which a drug-filled transparent cartridge 12 with a distal needle-penetrable septum 18 arranged and retained in place by a cartridge holder 46 attached to the proximal portion, the cartridge holder having a pair of opposed openings allowing a portion of the cartridge 12 to be inspected. Distal coupling means or needle mount 12 allows a needle assembly to be mounted in fluid communication with the cartridge interior. The cartridge is provided with a piston 20 driven by a piston rod forming part of the expelling mechanism and may for example contain an insulin, GLP-1 or growth hormone formulation. The cartridge 12 further comprises a rigid cylindrical body 16, and the piston 20 is slidably inserted in the cylindrical body 116.

A proximal-most rotatable dose setting member 40 serves to manually set a desired dose of drug shown in display window 44 and which can then be expelled when the button 48 is actuated. Depending on the type of expelling mechanism embodied in the drug delivery device, the expelling mechanism may comprise a torsion spring as in the shown embodiment which is strained during dose setting and then released to drive the piston rod when the release button is actuated. More specifically, during dose setting a drive member to which the spring is connected is rotated to a rotational position corresponding to the set dose, the drive member thereby being in an energized state. A scale drum with dose size numerals is coupled to the drive member such that the size of the currently set dose is shown in the display window, e.g. by means of a threaded connection with the housing. To prevent the drive member from rotating the dose setting mechanism is provided with a holding mechanism, which in the exemplary embodiment is in the form of a ratchet mechanism (not shown on figure). When the user desires to expel the set dose the button is actuated whereby the drive member is brought into engagement with the piston rod drive mechanism and the holding mechanism subsequently released.

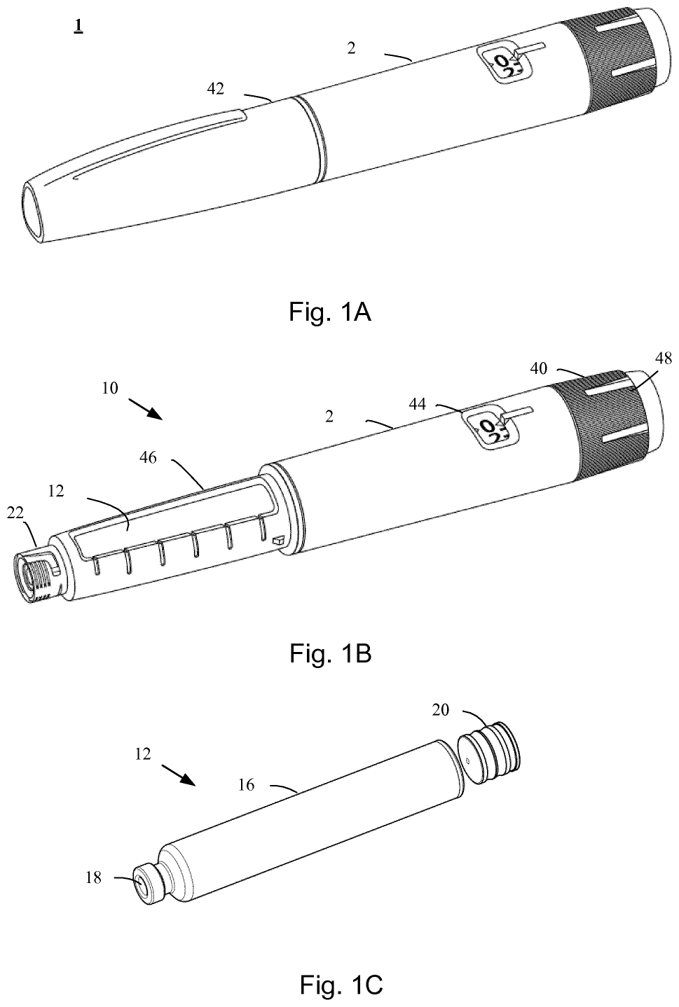

Although FIGS. 1A, 1B and 1C show a drug delivery device of the pre-filled type, i.e. it is supplied with a pre-mounted cartridge and is to be discarded when the cartridge has been emptied, in alternative embodiments the drug delivery device may be a durable device designed to allow a cartridge assembly to be replaced, e.g. in the form of a cartridge assembly comprising a cartridge mounted in a cartridge holder. Such an assembly may further be provided with a pre-mounted piston rod.

Preservatives are normally needed to prevent microbial or bacterial growth in drug formutations for extended or multiple use. In products from Novo Nordisk A/S, one or both of the preservatives phenol and m-cresol are used to ensure that minor microbial contaminations will not grow during the expected lifetime of a multi dosed injectable. However, phenol and m-cresol are toxic (which is required for them to work as intended) and may therefore as a side effect cause injection site reactions, or in some cases allergic reactions. This also means that additional restrictions applies to the selection of new protein/peptide drugs, since they are required to be preservative stabile, especially when the drug product is intended for daily or weekly use. Thus, it would in some cases be preferred to be able to reduce or omit the addition of preservatives to a given drug. It should be noted that substances that are regarded as preservatives may be added in lower amounts with the purpose of acting as stabilizer of the drug substance, e.g., insulin substances.

To ensure antimicrobial requirements can be met without adding preservatives to the drug itself, two major issues must be addressed. Firstly, it must be ensured that a contaminated needle or cannula cannot be inserted in the cartridge and introduce microbial contamination of the cartridge. Secondly, it must be ensured that backflow through the cannula is not possible which would introduce a risk of microbial contamination through backflow of body fluids from the user. This concept should not be confused with known arrangements in which preservative-filled reservoirs are provided to allow a subcutaneous needle to safely be used more than once, e.g. as disclosed in U.S. Pat. No. 3,354,881 and WO 2014/064100.

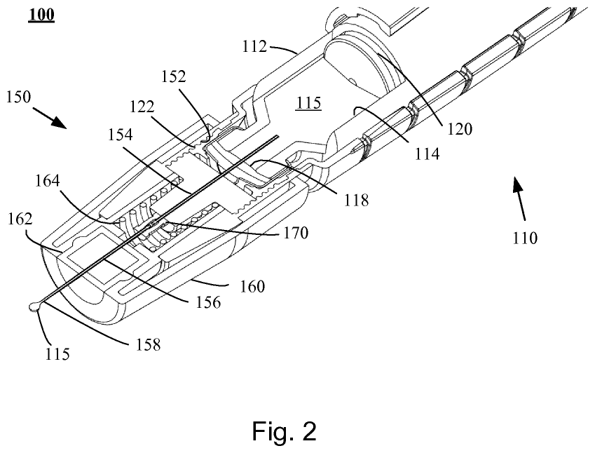

FIG. 2 shows an embodiment of a multi-use injection device 100 for multiple subcutaneous injections according to the present disclosure, for the purpose of describing the constructional features. The injection device is shown in an in-use configuration, wherein drug 115 is expelled through an injection needle. The injection device 100 comprises a device main portion 110 and a multi-use needle unit 150 adapted to prevent unintended introduction of living microorganisms into a reservoir 114 of the device main portion 110 during use of the injection device 100, and thereby promote bacteriostasis of a multiple-use drug preparation 115 during a use period, wherein multiple injections are to be applied. In FIG. 2, some of the components of the main portion 110 is not illustrated.

However, the main portion 110 of the embodiment 100 is similar to the main portion 10 shown in FIG. 1B.

The device main portion 110 comprises a cartridge 112 comprising the reservoir 114 with the multiple-use drug preparation 115 for subcutaneous injection. The multiple-use drug preparation comprises a mixture of substances, wherein the level of preservatives is to low to prevent growth of microorganisms, if such microorganisms are accidentally introduced into the reservoir with contaminations during use of the drug delivery device.

The cartridge 112 further comprises a rigid cylindrical body 116 with a distal end closed and sealed by a needle-piercable membrane 118, and a piston 120 slidably inserted in the cylindrical body 116.

The device main portion further comprises a needle mount 122 enabling the multi-use needle unit 150 to be mounted and establish fluid communication with the reservoir 114 by piercing the needle piercable membrane 118. The needle mount can be a thread connection or a bayonet coupling member, as illustrated on FIG. 1B as needle mount 22.

The device main portion further comprises, a dose setting member 40 adapted to set a dose to be expelled, an expelling mechanism comprising a piston drive member adapted to drive the piston 120 and thereby pressurize the reservoir 114 and expel the set dose through a mounted multi-use needle unit 150.

The multi-use needle unit 150 comprises a connecting member 152 for mounting the multi-use needle unit on the needle mount 122. The connecting member may be a thread or a bayonet coupling member. The needle unit also comprises a proximal hollow needle 154 adapted to penetrate or pierce the needle-piercable membrane 118, wherein the proximal needle 154 is adapted to establish fluid communication with the reservoir 114, when the needle unit 150 mounted on the needle mount.

The multi-use needle unit 150 further comprises a distal hollow expelling needle 156 with a sharp distal end 158, wherein the distal needle 156 is in fluid communication with the proximal needle 154 and adapted to be inserted into the subcutaneous tissue of a subject and to provide an outlet for expelling the drug 115 from the reservoir 114.

The multi-use needle unit 150 further comprises a movable shield 160 distally carrying a cleaning member 162. The shield 160 is adapted to be slidably operated between a first axial position and a second axial position relative to the distal end 158 of the distal needle 156 wherein: (i) the first axial position comprises the shield 160 extending distally to the distal end 158 of the distal needle 156, whereby the shield 160 covers the distal needle 156 and the distal end 158 is arranged in the cleaning member 162, and (ii) the second axial position comprises the distal end 158 extending distally to the shield 160, wherein the distal end 158 is exposed and ready for injection into the subcutaneous layer of a subject.

The cleaning member 162 is adapted to preserve the distal needle 156 in a sterile condition, and the shield 162 is arranged to automatically return to its first position following exposure of the distal needle 156 to the surroundings. Therefore, the cleaning member is adapted to sterilize any introduced contaminations during exposure of the distal needle 156.

The multi-use needle unit 150 further comprises a pressure-controlled valve 170. The valve is adapted to allow a drug flow from the reservoir 114 to the distal needle 156, in response to pressurization of the reservoir and the establishment of a pressure drop across the valve 170 in the a direction from the reservoir 114 towards the distal needle 156, wherein the pressure drop exceeds a first threshold.

The valve is further adapted to restrict a drug flow from the distal needle 156 to the reservoir 114, in response to the establishment of a pressure drop across the valve 170 in the direction from the distal needle towards the reservoir, wherein the pressure drop exceeds a second threshold.

The valve is further adapted to restrict diffusion from the distal needle 156 to the reservoir 114, when the pressure drop across the valve 170 is below the second threshold. In this way it can be prevented that contaminations diffuse from the distal needle to the reservoir.

The drug delivery device 100 further comprises an initial configuration and an in-use configuration. The initial configuration initial is the configuration of the device prior to a first exposure of the distal needle 156, wherein the needle unit 150 is mounted on the needle mount 122 of the device main portion 110 with the proximal needle 154 extending into the reservoir 114. In the in-use configuration, the needle unit 150 is mounted on the needle mount 122 of the device main portion 110 with the proximal needle 154 extending into the reservoir 114. The proximal needle 154 and the distal needle 156 are filled with drug from the reservoir 114, and the drug delivery device 100 is adapted to establish a vacuum in the reservoir 114 and a pressure drop across the valve 170 in the direction from the distal needle 156 towards the reservoir 114, in response to demounting the needle unit 150, whereby the primed proximal needle 154 is withdrawn from the reservoir 114. This functionality is inherent to a sealed and relatively rigid drug reservoir, wherein the withdrawal of a primed proximal needle expands the reservoir, and the effect of sucking the drug from the primed proximal and distal needle into the reservoir due the established vacuum is undesired. The effect is particularly undesired as the drug does not contain a sufficient level of preservatives to promote backteriostasis. Therefore, the needle unit is adapted for multi-use purposes, which takes away the requirement of changing needle. The pressure controlled valve and the cleaning member reduces the effect, if a vacuum accidentally should be established.

Hereby, the drug delivery device 100 is adapted to enable multiple injections, without accidentally introducing living microbial contaminations into the reservoir during use, by adapting the multi-use needle to: (i) sterilize contaminations introduced into the distal needle after each exposure, (ii) restrict diffusion and flow from the distal needle 156 to the reservoir 114), (iii), and by allowing the needle unit to remain mounted in the in-use configuration during the use period with multiple injections, without risking contamination of the multiple-use drug preparation (115) comprising the mixture of substances that do not prevent growth of microorganisms if the contaminations are accidentally introduced into the reservoir during use of the drug delivery device.

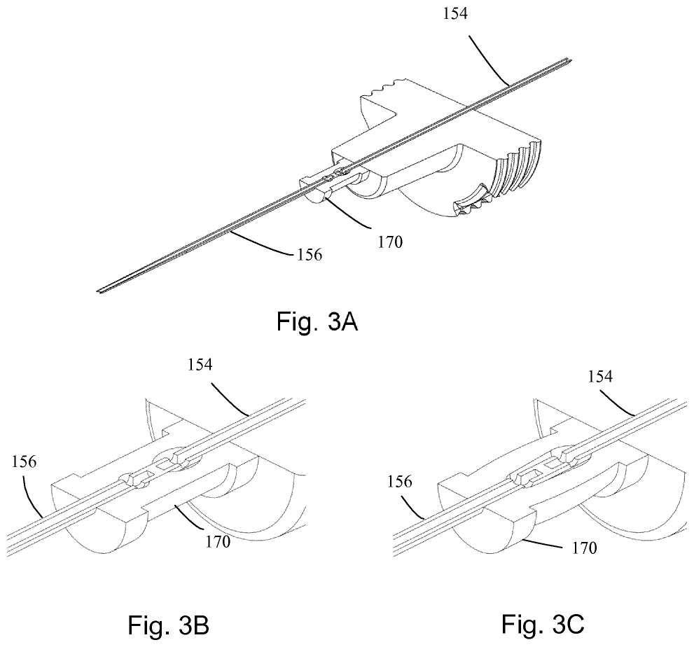

FIG. 3A illustrates the pressure regulated valve 170 in the embodiment illustrated on FIG. 2. FIG. 3B illustrates the valve 170 in a closed state, wherein the pressure in the reservoir 114 is below the first threshold, and wherein diffusion or flow is prevented from the distal needle 156 to the proximal needle 154. FIG. 3C illustrates the valve 170 in an open state, wherein the pressure in the reservoir exceeds the first threshold, and wherein the drug 115 can flow from the reservoir 114 to the distal needle 156, and out of the drug outlet at the distal end 158.

In an alternative embodiment of the injection device, the needle-unit and the device main portion are adapted to irremovably engage, when the needle unit is mounted on the needle mount. This functional feature can e.g. be obtained by providing a snap-lock between the needle unit and the device main portion. The functionality can also be obtained by actively locking the needle by a separate locking action after needle has been mounted, or by gluing the needle unit onto the device main portion. With this functionality it is avoided that vacuum is created in the reservoir due to accidental demounting of the needle-unit.

In an alternative embodiment of the injection device, the cleaning member 162 comprises a cleaning agent adapted to react with microbial contaminations introduced with the distal needle 156.

In some embodiments the cleaning agent comprises phenol and/or m-cresol.

In an alternative embodiment of the injection device, the cleaning member 162 is a solid.

In an alternative embodiment of the injection device, the cleaning member 162 comprises a cleaning chamber with a liquid cleaning agent.

In an alternative embodiment of the injection device, the injection device further comprises a protective cap 42, wherein the device main portion 10 further comprises a cap mount to receive the cap in a mounted state. The protective cap 42 is adapted to enclose the needle mount 22 in the mounted state.

In an alternative embodiment of the injection device, the protective cap 42 is further adapted to receive and enclose a multi-use needle unit 150 mounted on the needle mount.

In an alternative embodiment of the injection device, the needle unit 150 further comprises a spring 164 arranged and adapted to automatically return the shield 160 to its first axial position, in response to releasing a force urging the shield towards the second axial position. In an alternative embodiment the biasing means is obtained with compressed air in a pneumatic component.

In an alternative embodiment of the injection device, the device further comprises a disassembled configuration, wherein the device main portion 110 and the needle unit 150 are separated. In this way the main portion and the needle unit can be safely shelved after sterilization. The two components are then assembled just prior to its first use.

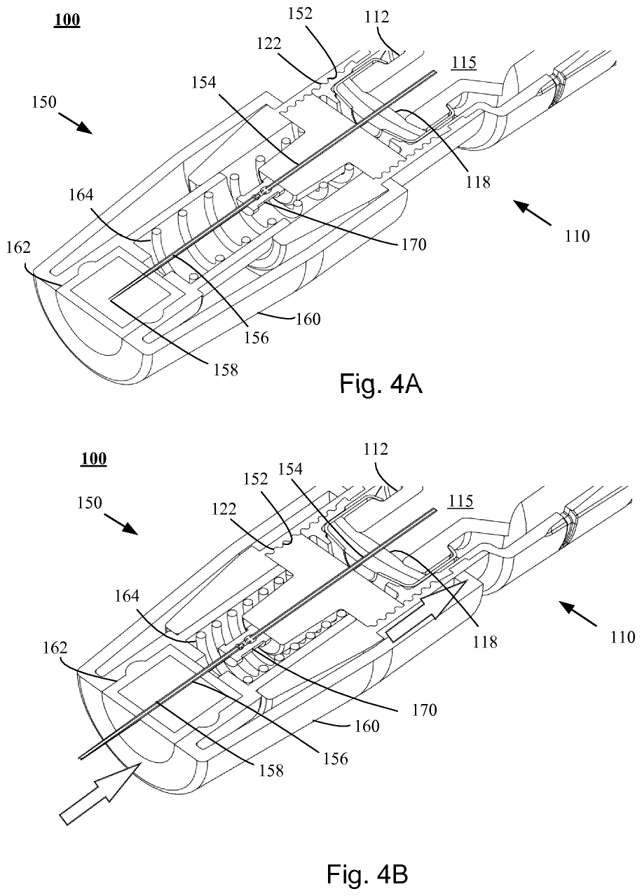

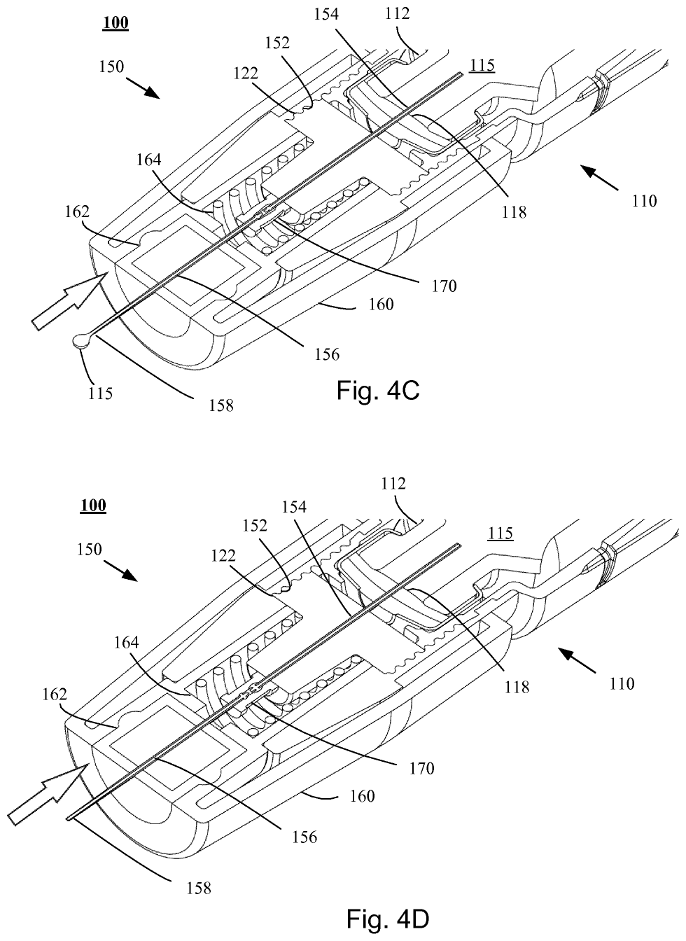

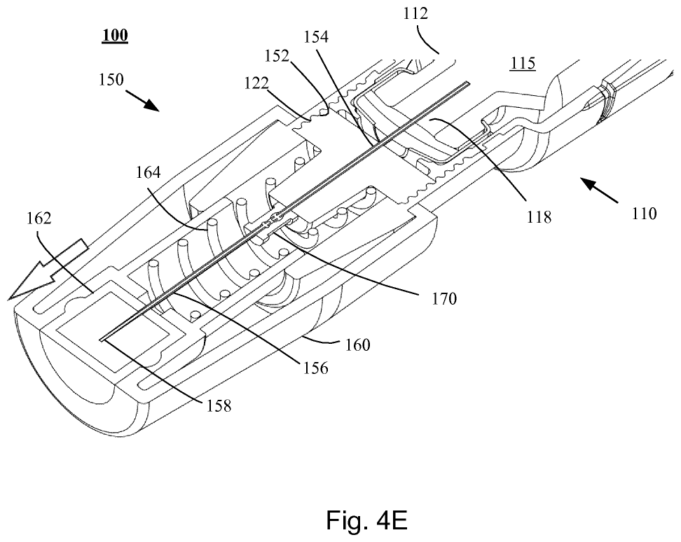

In a further aspect of the present disclosure is provided a method of using the multi-use injection device as disclosed, the method is illustrated on FIG. 4A to 4D. The injection device comprises the device main portion 110 and the multi-use needle unit 150 adapted to prevent unintended introduction of living microorganisms into the reservoir 114 of the device main portion during use of the injection device. Thereby the injection device is adapted to promote bacteriostasis of a multiple-use drug preparation 115 in the reservoir during a use period with multiple injections

The method comprises providing the injection device 100 in the initial assembled configuration, wherein, as illustrated FIG. 4A, the needle unit is mounted on the needle mount of the device main portion 110 with the proximal needle 154 extending into the reservoir 114.

The method further comprises moving the shield 160 and the cleaning member 162 from the first axial position to the second axial position, as illustrated on FIG. 4B. The arrow indicates the application of an external force pushing the shield against the force of the biasing means. By changing the shield to the second axial position, the distal end 158 extends distally to the shield 162, wherein the distal end 158 is exposed to contaminations in the surroundings and is ready for the initial priming and/or expelling an amount of drug.

The method further comprises pressurizing the reservoir 114, wherein the effect is illustrated in FIG. 4C. By the pressurization an initial amount of drug is expelled, whereby the proximal needle 154 and the distal needle 156 are filled with drug 115, whereby the injection device 100 is in the in-use configuration. As the pressure in the reservoir falls below a first threshold, the expulsion is stopped, which is illustrated on FIG. 4D.

The method further comprises moving the shield 160 and the cleaning member from the second axial position to the first axial position, as illustrated in FIG. 4E, and thereby positioning the distal end 158 of the distal tip in the cleaning member again. Hereby, and in this position, the cleaning member sterilizes any introduced contamination.

For handling the injection device to provide further expulsions during use, without contaminating the reservoir, the method further comprises moving the shield and the cleaning member from the first axial position to the second axial position, whereby the distal end extends distally to the shield, wherein the distal end is exposed to contaminations in the surroundings and is ready for expelling an amount of drug. The method further comprises pressurizing the reservoir and thereby expelling an amount of drug, whereby the proximal and the distal needle are filled with drug, and whereby the expelling reliefs the pressure in the reservoir until the pressure reaches the first threshold, wherein the pressure controlled valve closes and restricts diffusion and flow from the distal needle 156 to the reservoir 114. The method further comprises, moving the shield 160 and the cleaning member 162 from the second axial position to the first axial position, and thereby positioning the distal end 158 of the distal needle 156 in the cleaning member 162, whereby the cleaning member 162 sterilizes any introduced microbial contamination.

Hereby the method provides multiple injections, without accidentally introducing living microbial contamination into the reservoir during use, by (i) sterilizing microbial contaminations introduced into the distal needle after each exposure, (ii) restricting diffusion and flow from the distal needle 156 to the reservoir 114, (iii), and performing multiple injections, wherein the needle unit 150 remains mounted in the in-use configuration during the use period with multiple injections.

Alternatively, the method of using the multi-use injection device according to present disclosure, the method comprises:
sterilizing microbial contaminations introduced into the distal needle after each exposure,
restricting diffusion and flow from the distal needle (156) to the reservoir (114), and
performing multiple injections, wherein the needle unit (150) remains mounted in the in-use configuration during the use period with multiple injections.

In a further embodiment, the needle-unit 150 and the device main portion 110 are adapted to irremovably engage, when the needle unit is mounted on the needle mount, wherein the method further comprises mounting the needle unit 150 on the needle mount 122 and thereby permanently engaging the needle unit 150 to the device main portion 110. In a further embodiment the needle unit is snap-locked onto the device main portion.

In the above description of exemplary embodiments, the different structures and means providing the described functionality for the different components have been described to a degree to which the concept of the present invention will be apparent to the skilled reader. The detailed construction and specification for the different components are considered the object of a normal design procedure performed by the skilled person along the lines set out in the present specification.

1. A multi-use injection device for multiple subcutaneous injections, wherein the injection device comprises a device main portion and a multi-use needle unit adapted to prevent unintended introduction of living microorganisms into a reservoir of the device main portion during use of the injection device, and thereby promote bacteriostasis of a multiple-use drug preparation during a use period with multiple injections:
the device main portion (110) comprises:
a cartridge comprising the reservoir with the multiple-use drug preparation for subcutaneous injection, wherein the multiple-use drug preparation comprises a mixture of substances that do not prevent growth of microorganisms if accidentally introduced into the reservoir with contaminations during use of the drug delivery device,
wherein the cartridge further comprises a rigid cylindrical body with a distal end closed and sealed by a needle-piercable membrane, and a piston slidably inserted in the cylindrical body,
a needle mount enabling the multi-use needle unit to be mounted and establish fluid communication with the reservoir by piercing the needle piercable membrane,
dose setting member adapted to set a variable dose to be expelled after each injection or a fixed dose mechanism to set a fixed dose to be expelled after each injection, an expelling mechanism comprising a piston drive member adapted to drive the piston and thereby pressurize the reservoir (444) and expel the set dose through a mounted multi-use needle unit;
wherein the multi-use needle unit comprises:
a connecting member for mounting the multi-use needle unit on the needle mount,
a proximal hollow needle adapted to penetrate the needle-piercable membrane, wherein the proximal needle is adapted to establish fluid communication with the reservoir, [when the needle unit is mounted on the needle mount],
a distal hollow expelling needle with a sharp distal end, wherein the distal needle is in fluid communication with the proximal needle and adapted to be inserted into the subcutaneous tissue of a subject and to provide an outlet for expelling the drug from the reservoir,
a movable shield distally carrying a cleaning member, wherein the shield is adapted to be slidably operated between a first and a second axial position relative to the distal end of the distal needle, wherein (i) the first axial position comprises the shield extending distally to the distal end of the distal needle, whereby the shield covers the distal needle and the distal end is arranged in the cleaning member, wherein (ii) the second axial position comprises the distal end extending distally to the shield, wherein the distal end is exposed and ready for injection into the subcutaneous layer of a subject,
wherein the cleaning member is adapted to preserve the distal needle in a sterile condition,
wherein the shield is arranged to automatically return to its first position following exposure of the distal needle, wherein the cleaning member is adapted to sterilize any introduced contaminations during exposure of the distal needle,
a pressure-controlled valve, wherein the valve is adapted to:
allow a drug flow from the reservoir to the distal needle, in response to pressurization of the reservoir and the establishment of a pressure drop across the valve in a direction from the reservoir towards the distal needle, wherein the pressure drop exceeds a first threshold,
restrict a drug flow from the distal needle to the reservoir, in response to the establishment of a pressure drop across the valve in the direction from the distal needle towards the reservoir, wherein the pressure drop exceeds a second threshold,
restrict diffusion from the distal needle to the reservoir, when the pressure drop across the valve is below the second threshold,
wherein the drug delivery device comprises:
an initial assembled configuration prior to a first exposure of the distal needle, wherein the needle unit is mounted on the needle mount of the device main portion with the proximal needle extending into the reservoir,
an in-use configuration, wherein the needle unit is mounted on the needle mount of the device main portion with the proximal needle extending into the reservoir, wherein the proximal needle and the distal needle are filled with drug from the reservoir, and wherein the drug delivery device is adapted to establish a vacuum in the reservoir and a pressure drop across the valve in the direction from the distal needle towards the reservoir, in response to demounting the needle unit and thereby withdrawing the primed proximal needle from the reservoir,
wherein the drug delivery device is adapted to enable multiple injections, without accidentally introducing living microbial contaminations into the reservoir during use, by adapting the multi-use needle to: (i) sterilize contaminations introduced into the distal needle after each exposure, (ii) restrict diffusion and flow from the distal needle to the reservoir, (iii), and by allowing the needle unit to remain mounted in the in-use configuration during the use period with multiple injections, without risking contamination of the multiple-use drug preparation comprising the mixture of substances that do not prevent growth of microorganisms if the contaminations are accidentally introduced into the reservoir during use of the drug delivery device.

2. The injection device according to claim 1, wherein the needle-unit and the device main portion are adapted to irremovably engage, when the needle unit is mounted on the needle mount.

3. The injection device according to claim 1, wherein the cleaning member comprises a cleaning agent adapted to react with microbial contaminations introduced with the distal needle.

4. The injection device according to claim 1, wherein the cleaning member is a solid.

5. The injection device according to claim 1, wherein the cleaning member comprises a cleaning chamber with a liquid cleaning agent.

6. The injection device according to claim 1, wherein the injection device further comprises a cap, wherein the device main portion further comprises a cap mount to receive the cap in a mounted state, wherein the cap is adapted to enclose the needle mount in the mounted state.

7. The injection device according to claim 6, wherein the cap is further adapted to receive and enclose a multi-use needle unit mounted on the needle mount.

8. The injection device according to claim 1, wherein the needle unit further comprises a spring arranged and adapted to automatically return the shield to its first axial position, in response to releasing a force urging the shield towards the second axial position.

9. The injection device according to claim 1, wherein the injection device further comprises a disassembled configuration, wherein the device main portion and the needle unit are separated.

10. A method of using the multi-use injection device according to claim 1, wherein the injection device comprises the device main portion and the multi-use needle unit adapted to prevent unintended introduction of living microorganisms into a reservoir of the device main portion during use of the injection device, and thereby promote bacteriostasis of a multiple-use drug preparation during a use period with multiple injections, wherein the method comprises:
sterilizing microbial contaminations introduced into the distal needle after each exposure,
restricting diffusion and flow from the distal needle to the reservoir, and
performing multiple injections, wherein the needle unit remains mounted in the in-use configuration during the use period with multiple injections.

11. The method according to claim 10, wherein the needle-unit and the device main portion are adapted to irremovably engage, when the needle unit is mounted on the needle mount, wherein the method further comprises mounting the needle unit on the needle mount and thereby permanently engaging the needle unit to the device main portion.

12. The method using the multi-use injection device according to claim 1, wherein the needle-unit and the device main portion are adapted to irremovably engage, when the needle unit is mounted on the needle mount, wherein the method comprises mounting the needle unit on the needle mount and thereby permanently engaging the needle unit to the device main portion.

13. The method of using the multi-use injection device according to claim 1, wherein the injection device comprises the device main portion and the multi-use needle unit is adapted to prevent unintended introduction of living microorganisms into a reservoir of the device main portion during use of the injection device, and thereby promote bacteriostasis of a multiple-use drug preparation during a use period with multiple injections, wherein the method comprises:
providing the injection device in the initial assembled configuration, wherein the needle unit is mounted on the needle mount of the device main portion with the proximal needle extending into the reservoir,
moving the shield and the cleaning member from the first axial position to the second axial position, whereby the distal end extends distally to the shield, wherein the distal end is exposed to contaminations in the surroundings and is ready for the initial priming and expelling an amount of drug,
pressurizing the reservoir and thereby expelling an initial amount of drug, whereby the proximal needle and the distal needle are filled with drug, whereby the injection device is in the in-use configuration
moving the shield and the cleaning member from the second axial position to the first axial position, and thereby positioning the distal end of the distal tip in the cleaning member, whereby the cleaning member sterilizes any introduced contamination,
for handling the injection device to provide further expulsions during use, the method further comprises:
a) moving the shield and the cleaning member from the first axial position to the second axial position, whereby the distal end extends distally to the shield, wherein the distal end is exposed to contaminations in the surroundings and is ready for expelling an amount of drug,
b) pressurizing the reservoir and thereby expelling an amount of drug, whereby the proximal and the distal needle are filled with drug, and whereby the expelling releases the pressure in the reservoir until the pressure reaches the first threshold, wherein the pressure controlled valve closes and restricts diffusion and flow from the distal needle to the reservoir,
c) moving the shield and the cleaning member from the second axial position to the first axial position, and thereby positioning the distal end of the distal needle in the cleaning member, whereby the cleaning member sterilizes any introduced contamination,
whereby the method provides multiple injections, without accidentally introducing living microbial contamination into the reservoir during use, by (i) sterilizing microbial contaminations introduced into the distal needle after each exposure, (ii) restricting diffusion and flow from the distal needle to the reservoir, (iii), and performing multiple injections, wherein the needle unit remains mounted in the in-use configuration during the use period with multiple injections.
